(19) United States
(12) Patent Application Publication
(10) Pub. No.: US 2011/0229527 A1
(43) Pub. Date: Sep. 22, 2011

(54) DIRECTLY COMPRESSIBLE HIGH FUNCTIONALITY GRANULAR DIBASIC CALCIUM PHOSPHATE BASED CO-PROCESSED EXCIPIENT

(76) Inventors: Nandu Deorkar, Cedar Knolls, NJ (US); James Farina, Nazareth, PA (US); Liliana Miinea, Whitehall, PA (US); Sameer Randive, Thane-West (IN)

(21) Appl. No.: 12/998,655

(22) PCT Filed: Nov. 13, 2009
(86) PCT No.: PCT/US2009/064289

§ 371 Date: May 16, 2011

Related U.S. Application Data
(60) Provisional application No. 61/116,349, filed on Nov. 20, 2008.

(51) Int. Cl. A61K 9/14; A61K 47/04; A61K 47/38; D04H 1/64; B29C 59/02

(52) U.S. Cl. 424/400; 514/769; 264/13; 264/109

(57) ABSTRACT

An improved excipient comprising substantially homogeneous particles of a compressible, high functionality granular dibasic calcium phosphate based excipient is provided. The improved excipient comprises dibasic calcium phosphate, a binder and a disintegrant, and is formed by spraying a homogeneous slurry of the components. The improved excipient provides enhanced flowability/good flow properties, an increased API loading and blendability and higher compactibility as compared to the individual components, and as compared to excipients formed from the same materials by conventional methods.

The improved excipient has strong intraparticle bonding bridges between the components, resulting in a unique structural morphology including significant open structures or hollow pores. The presence of these pores provides a surface roughness that is the ideal environment for improved blending with an API.

### BACKGROUND OF INVENTION

The most commonly employed means to deliver drug substances is the tablet, typically obtained through the compression of appropriately formulated excipient powders. Tablets should be free of defects, have the strength to withstand mechanical shocks, and have the chemical and physical stability to maintain physical attributes over time and during storage. Undesirable changes in either chemical or physical stability can result in unacceptable changes in the bioavailability of the drug substance. In addition, tablets must be able to release the drug substance in a predictable and reproducible manner. The present invention relates to a novel excipient for use in the manufacture of pharmaceutical solid dosage forms such as tablets. The novel excipient is advantageously combined with at least one drug substance, hereinafter active pharmaceutical ingredient (API), and formed into tablets using a direct compression manufacturing method.

In order to successfully form tablets, the tableting mixture must flow freely from a feeder hopper into a tablet die, and be suitably compressible. Since most APIs have poor flowability and compressibility, APIs are typically mixed with varying proportions of various excipients to impart desired flow and compressibility properties. In typical practice, a compressible mixture is obtained by blending an API with excipients such as diluents/fillers, binders/adhesives, disintegrants, glidants/flow promoters, colors, and flavors. These materials may be simply blended, or may be wet or dry granulated by conventional methods. Once mixing is complete, a lubricating excipient is typically added and the resulting material compressed into tablets.

Unfortunately, there are few general rules regarding excipient compatibility with particular APIs. Therefore, when developing tablet formulations to meet particular desired characteristics, pharmaceutical scientists typically must conduct an extensive series of experiments designed to determine which excipients are physically and chemically compatible with a specific API. Upon completion of this work, the scientist deduces suitable components for use in one or more trial compositions.

A commonly used excipient is microcrystalline cellulose (MCC). MCC has a suitable compressibility and is chemically inert with many APIs. However, the functional groups on MCC have the potential of reacting with certain API functional groups. One potential substitute for MCC is dibasic calcium phosphate (DCP), which is chemically inert with most APIs. Dibasic calcium phosphate is the most common inorganic salt used as pharmaceutical excipient. However, the use of DCP has two major disadvantages. First, DCP has an extremely low compressibility, making it difficult to form suitable tablets by direct compression. Further, DCP is physically abrasive, lending an undesirable mouth feel to tablets, as well as leading to increased wear and tear of tableting punches.

Attempts have been made to produce improved DCP formulations. U.S. Pat. No. 4,675,188 to Chu et al. discloses a granular directly compressible anhydrous dibasic calcium phosphate excipient which purports to have a particle size sufficient for efficient direct compression tableting. According to the disclosure, dibasic calcium phosphate is dehydrated, and then granulated with a binder. The resulting product is purportedly a granular anhydrous dibasic calcium phosphate, characterized in that at least 90 percent of the particles are larger than 44 microns. This granular product purports to improve over commonly used precipitated anhydrous dibasic calcium phosphate, which is a fine, dense powder that must be agglomerated with a binder such as starch before it can be used in direct compression tableting. The process disclosed in Chu et al. consists of coating anhydrous calcium phosphate with starch or another binder, purportedly resulting in binding of calcium phosphate particles to each other forming large particles. However, this granulated product is not a universal excipient, in that it lacks other necessary excipients, such as disintegrants, that are necessary to produce a pharmaceutically acceptable tablet after compression.

There is therefore a need for a universal excipient including an improved DCP formulation that provides sufficient compressibility and reduces abrasiveness.

### SUMMARY OF INVENTION

An illustrative aspect of the present invention is a composition comprising about 75% to about 98% dibasic calcium phosphate; about 1% to about 10% at least one binder; and about 1% to about 20% at least one disintegrant.

Another illustrative aspect of the present invention is an excipient comprising about 75% to 98% DCP, about 1% to about 10% at least one binder, and 1% to about 20% at least one disintegrant, wherein the excipient is formed by spraying an aqueous slurry comprised of DCP, binder and disintegrant. The dibasic calcium phosphate, binder and disintegrant form substantially homogeneous spherical particles in which the dibasic calcium phosphate, binder and disintegrant are indistinguishable when viewed with an SEM.

Yet another illustrative aspect of the present invention is a method of making an excipient. The method comprises forming a dibasic calcium phosphate slurry; forming a binder slurry; and forming a disintegrant slurry; homogenizing the dibasic calcium phosphate slurry and the disintegrant slurry to form a DCP/disintegrant slurry; adding the binder slurry to the DCP/disintegrant slurry; and spray dry granulating the final slurry to form homogeneous spherical particles of excipient. The dibasic calcium phosphate, binder and disintegrant are indistinguishable when viewed with an SEM, thereby forming substantially homogeneous spherical particles.

Still another illustrative aspect of the present invention is a method of making an excipient. The method comprises forming a dibasic calcium phosphate slurry; forming a hydroxypropyl methylcellulose slurry; forming a cross-linked polyvinylpyrrolidone (CPVD) slurry; homogenizing the dicalcium phosphate slurry and the cross-linked polyvinylpyrrolidone slurry to form a DCP/CPVD slurry; adding the hydroxypropyl methylcellulose slurry to the DCP/CPVD slurry; and spray dry granulating the final slurry to form homogeneous spherical particles of excipient. The dibasic calcium phosphate, hydroxypropyl methylcellulose and cross-linked polyvinylpyrrolidone are indistinguishable when viewed with a SEM, thereby forming substantially homogeneous particles.

Still another illustrative aspect of the present invention is a pharmaceutical tablet comprising at least one active pharmaceutical ingredient and an excipient of substantially homogeneous particles including dibasic calcium phosphate, at least one binder and at least one disintegrant.

Still a further illustrative aspect of the present invention is a method of making a pharmaceutical tablet comprising mixing at least one active pharmaceutical ingredient with an excipient of substantially homogeneous particles including dibasic calcium phosphate, at least one binder and at least one disintegrant to form a mixture; and compressing the mixture to form a tablet.

### BRIEF DESCRIPTION OF DRAWINGS

FIG. 1 is an illustration of SEM micrographs of the improved excipient of the present invention produced according to Example 1.

FIG. 2 is an illustration of SEM micrographs of the granular material produced according to Example 3

FIG. 3 is an illustration of SEM micrographs of Dibasic Calcium Phosphate commercially available from Malinckrodt Baker, Inc.

FIG. 4 is an illustration of SEM micrographs of Dibasic Calcium Phosphate commercially available from Rhodia, Inc.

FIG. 5 is an illustration of SEM micrographs of Dibasic Calcium Phosphate commercially available from Nitika Chemicals.

FIG. 6 is an illustration of the dissolution profile for Diclofenac Sodium from tablets prepared at 5000 lbs-force according to Example 9.

### DETAILED DESCRIPTION

There is provided an improved excipient comprising substantially homogeneous spherical particles of a compressible, high functionality granular dibasic calcium phosphate based excipient. The improved excipient provides enhanced flowability/good flow properties, an increased API loading and blendability, and higher compactibility as compared to the individual components, and as compared to excipients formed from the same materials by conventional methods. The improved excipient is especially beneficial for use with APIs that have the potential to react with other diluents/fillers.

The improved excipient has strong intraparticle bonding bridges between the components, resulting in a unique structural morphology including significant open structures or hollow pores. The presence of these pores provides a surface roughness that is the ideal environment for improved blending with an API. Excellent blendability is an essential characteristic of an excipient as it allows tablets to be produced that contain a uniform amount of the API. Additionally, this improved excipient includes the necessary excipients, except for the optional lubricant, that are required to produce a pharmaceutically acceptable tablet.

The improved excipient is engineered to have particle size and density that greatly improves compressibility as compared to conventional DCP. This results in the improved excipient being directly compressible, complete, and universal excipient for making pharmaceutical tablets. The excipient is considered complete since it includes a diluent, a binder and a disintegrant, and is considered universal since it is compatible with a variety of APIs. The components and physical characteristics of the improved excipient were carefully chosen and optimized to ensure its use in formulating a wide range of APIs.

The universality of this excipient overcomes the need for the traditional time consuming approach to formulation development, wherein the scientist develops a custom blend of various excipients to optimize flowability and compressibility for the particular API. It was unexpectedly discovered that the disclosed composition and process of making the improved excipient provides a substantially homogeneous, strong spherical particle having high increased porosity that provides good flowability and good compactibility. The improved excipient typically has an aerated bulk density of about 0.5 g/cc.

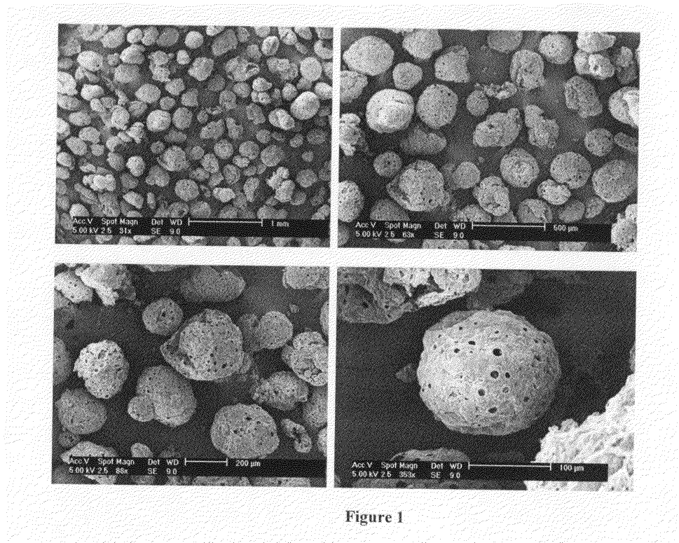

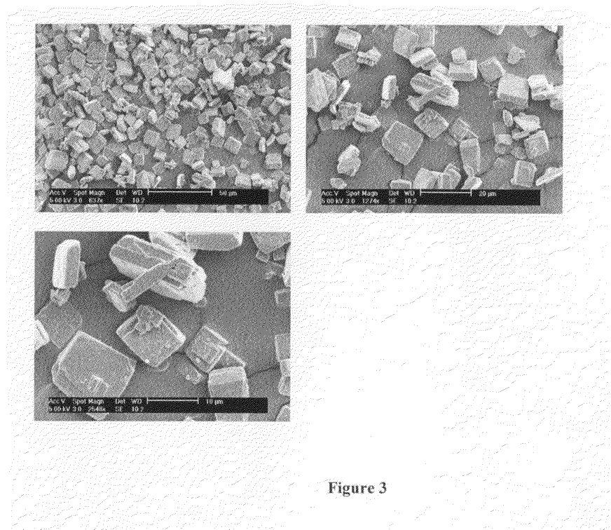

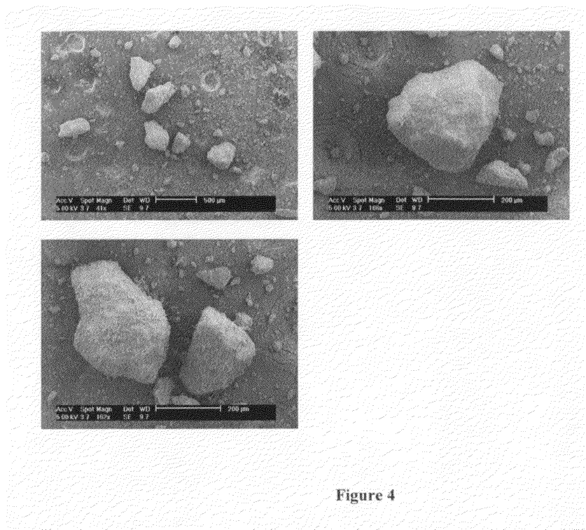

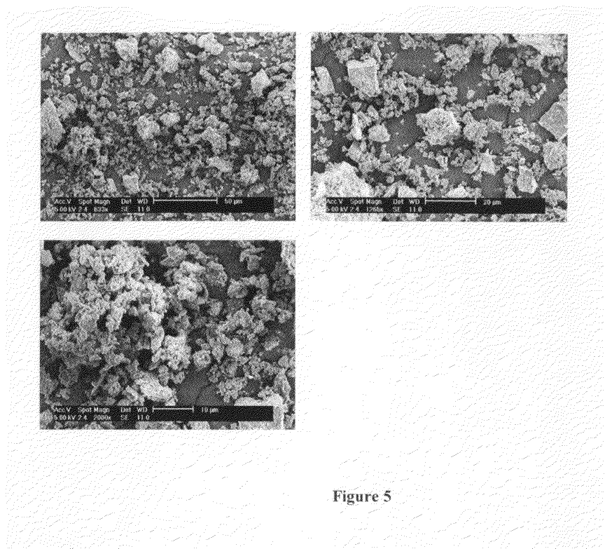

Unprocessed DCP has a parallelepiped or irregular shape when viewed under SEM (as illustrated in FIGS. 3, 4 and 5). The particle morphology of the improved excipient disclosed herein is unexpectedly unique as a substantially homogeneous spherical structure with holes or pores and hollow portions in the particles that can improve API loading capacity. As is illustrated in FIG. 1, the term substantially homogeneous is meant herein to denote a structure in which the individual components cannot be distinguished under SEM scan.

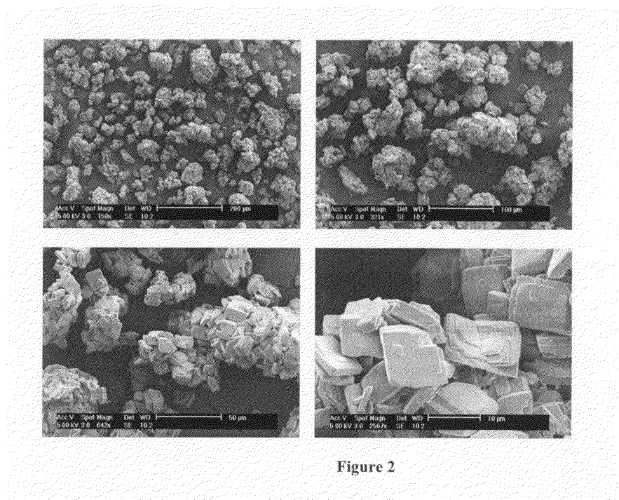

The granules formed in the traditional and other disclosed processes are seen as a simple bonding of particles into irregularly shaped granules produced by agglomeration of distinct particles. This is seen in Example 3 and in FIG. 2. It is common for these agglomerated particles to separate into the distinct components during transport or rough handling. The continuous spherical particles of the improved excipient, while including hollow portions, are unexpectedly robust and are not friable during handling and processing.

In the present invention, DCP is processed in combination with a polymeric binder and a cross-linked hygroscopic polymer to produce spherical particles having high porosity and strong intraparticle binding. The polymeric binder is selected from the class of cellulosic polymers or organic synthetic polymers having thermal stability at about 80° C. to about 120° C., dynamic viscosity in the range of about 2 mPa to about 50 mPa for a water solution of about 0.5% to about 5% wt/vol, water solubility in the range of about 0.5% to about 5% wt/vol and providing a surface tension in the range of about 40 dynes/cm to about 65 dynes/cm for about 0.5% to about 5% wt/vol water solution. Preferred binders from this class include hydroxypropyl methylcellulose, hydroxyethyl cellulose, hydroxypropyl cellulose, sodium carboxymethyl cellulose, and polyvinyl alcohol-polyethylene glycol graft copolymer and vinylpyrrolidone-vinyl acetate copolymer. Presently preferred is hydroxypropyl methylcellulose (HPMC). The cross-linked hygroscopic polymer disintegrant is preferably crospovidone (CPVD). As is seen in FIG. 1, the processed particles are a substantially homogeneous composition of spheres with porous portions leading to at least partially hollow portions of the spheres. The granules are produced by the actual physical binding of the slurry mixture that becomes distinct particles when ejected out of the nozzle. The porosity and hollow portions result in improved API loading and blendability.

Example 1 illustrates an improved excipient formulation, while Examples 2 and 3 illustrate conventional (High shear wet granulation) formulations of the same component percentages, and Example 4 provides conventional powder blends.

Example 5 compares friability of the granules prepared according to the present invention as per Example 1 and the friability of granules prepared by conventional method as per Example 3. While the percentage of fines remains virtually unchanged for the improved excipient, the percentage of fines for the excipient prepared by conventional method increases by about 70%. This indicates that the improved excipient has very strong particles that can sustain rough handling.

The improved excipient has excellent flowability. In general, when particle flow is poor, additional glidants such as silicon dioxide are added to improve flow. If the powder flow is not sufficient, poor tablet productivity will result. Characterization of the improved excipient particles by the Carr method, well know in the art, showed a flowability index that exceeds 85, where a flowability index over 70 indicates good flowability. As is seen in Example 6, a Hosokawa powder tester, a test instrument that measures powder characteristics using a set of automated tests using the Carr method was used to determine that the improved excipient has a very good flowability when compared to the excipient prepared by conventional method. The flowability of a powder blend (Example 4b) of the same components as the ones used to prepare the improved excipient is extremely poor, being very difficult to be measured.

Example 7 compares the compressibility index and Hausner ratios of Example 1, Example 3 and Example 4b excipients. A value of 20-21% or less for the Carr's compressibility index and a value below 1.25 for the Hausner ratio indicate a material with good flowability. Example 1 material has the best flowability when compared to Example 3 and Example 4b.

Disintegration times and hardness values of tablets produced with the improved excipient as compared to conventional DCP formulations is illustrated in Example 8. The improved excipient produced tablets with acceptable hardness while Example 3 and 4b excipients produce soft tablets. This shows that Example 1 excipient has better compressibility than Example 3 and 4b excipients.

The process disclosed herein is a novel form of the spray drying granulation process. The new process consists of mixing each component with deionized water to form a DCP slurry, a binder slurry and a disintegrant slurry. The DCP slurry and the disintegrant slurry are mixed together first, and then the binder slurry is added. The homogenization process is carried out to bring the two insoluble components, DCP and a disintegrant, in contact with each other and bound in close association with a viscous binder slurry, for example hydroxypropyl methylcellulose. The evaporation of water at a high rate at high temperatures of 120° C. or more and the local action of HPMC holding all components together produces particle with unique shape and morphology. Illustrative non-limiting examples of this method are disclosed in Example 1.

In contrast, the traditional wet granulation method presented in Examples 2 and 3 consisted of dry mixing of the three components and the addition of a liquid binder (water). FIG. 2 illustrates the granular material obtained using the composition components of the present invention processed by the traditional wet granulation method. The material produced from the conventional high shear wet granulation process consisted of irregular shape friable particles that did not perform as well as the product formed by the present invention. Compressibility decreased, resulting in a 2.25 times decrease in the hardness of the placebo tablets pressed from the conventionally produced material as compared to the improved excipient according to Example 1, see Example 8. The particle morphology is composed of irregular particles bonded together by simple intergranular bridges, as seen in FIG. 2.

The components of the improved excipient are processed by an improved wet homogenization/spray dry granulation method. In this process, a slurry is formed of two water insoluble components (typically with a large difference in composition between the two water insoluble components) and a third water soluble component. The resulting slurry is granulated to a desired particle size, typically greater than about 50 μm, preferably about 50 μm to about 250 μm, and more preferably about 90 μm to about 150 μm.

The improved excipient is formed by converting the DCP into a slurry with deionized water; forming a binder slurry; and forming a disintegrant slurry; homogenizing the dibasic calcium phosphate slurry and the disintegrant slurry to form a DCP/disintegrant slurry; adding the binder slurry to the DCP/disintegrant slurry; and spray dry granulating the final slurry to form homogeneous spherical particles of excipient. In an illustrative embodiment, the excipient is formed from about 75% to about 98% DCP, in combination with about 1% to about 10% binder and about 1% to about 20% disintegrant. In a preferred embodiment, the excipient is formed from about 80% to about 90% DCP, about 2% to about 8% binder and about 3% to about 12% disintegrant. In a more preferred embodiment, the excipient is formed from about 85% to about 93% DCP, about 2% to about 5% binder and about 10% at least disintegrant.

The use of the improved excipient will reduce formulation development to a series of blending steps: blending of an API with the improved excipient (which contains the essential components of tablet formulation, diluent, binder and disintegrant) and optionally a lubricant.

APIs refers to one or more compounds that have pharmaceutical activity, including therapeutic, diagnostic or prophylactic utility. The pharmaceutical agent may be present in an amorphous state, a crystalline state or a mixture thereof. The active ingredient may be present as is, taste masked, or coated for enteric or controlled release. Suitable APIs are limited only in that they are compatible with DCP and the other excipient components. This allows the present invention improved DCP excipient to be utilized with APIs that have the potential of chemical reaction with other fillers/diluents. The blending process will be followed by pressing high quality tablets by direct compression.

Illustrative suitable APIs that can be used with the present invention include, but are not limited to: Antiviral agents, including but not limited to acyclovir, famciclovir; anthelmintic agents, including but not limited to albendazole; lipid regulatihg agents, including but not limited to atorvastatin calcium, simvastatin; angiotensin converting enzyme inhibitor including but not limited to benazepril hydrochloride, fosinopril sodium; angiotensin II receptor antagonist including but not limited to irbesartan, losartan potassium, valsartan; antibiotic including but not limited to doxycycline hydrochloride; antibacterial including but not limited to linezolid, metronidazole, norfloxacin; antifungal including but not limited to terbinafine; antimicrobial agent including but not limited to ciprofloxacin, cefdinir, cefixime; antidepressant, including but not limited to bupropione hydrochloride, fluoxetine; anticonvulsant including but not limited to carbamazepine; antihistamine including but not limited to loratadine; antimalarial including but not limited to mefloquine; antipsychotic agent including but not limited to olanzapine; anticoagulant including but not limited to warfarin; α-andrenergic blocking agent including but not limited to carvedilol, propranolol; selective H1-receptor antagonist including but not limited to cetirizine hydrochloride, fexofenadine; histamine H2-receptor antagonist including but not limited to cimetidine, famotidine, ranitidine hydrochloride, ranitidine; anti anxiety agent including but not limited to diazepam, lorazepam; anticonvulsants including but not limited to divalproex sodium, lamotrigine; inhibitor of steroid Type II 5α-reductase including but not limited to finasteride; actetylcholinesterase inhibitor including but not limited to galantamine; blood glucose lowering drug including but not limited to glimepiride, glyburide; vasodilator including but not limited to isosorbide dinitrate; calcium channel blocker including but not limited to nifedipine; gastric acid secretion inhibitor including but not limited to omeprazole; analgesic/antipyretics including but not limited to aspirin, acetaminophen, ibuprofen, naproxen sodium, oxycodone, oxymorphone, hydrocodone, hydromorphone, morphine, and codeine; erectile dysfunction including but not limited to sildenafil; diuretic including but not limited to hydrochlorothiazide; vitamins including but not limited to vitamin A, vitamin B1, vitamin B2, vitamin B6, vitamin B12, vitamin C, vitamin D, vitamin E, vitamin K or folic acid.

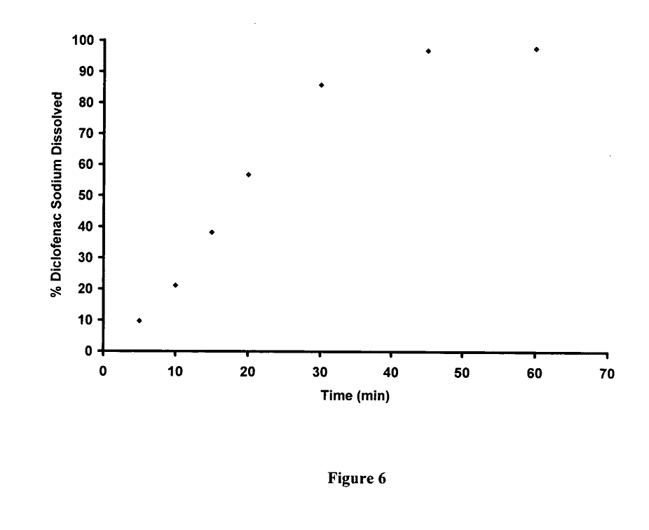

A non-limiting example of a tablet comprising the improved excipient and an API, specifically diclofenac sodium, is prepared in Example 9. The immediate release tablets of Example 9 provided a disintegration time of less than about 30 minutes. A dissolution profile is illustrated in FIG. 6.

Therefore, the composition and processing steps disclosed herein produce an improved excipient exhibiting novel final particle morphology, unexpectedly improved compressibility over unprocessed DCP, as well as decreased abrasiveness.

### Example 1

Preparation of dibasic calcium phosphate-5% hydroxypropyl methylcellulose—crospovidone excipient according to the present invention:

The excipient consists of dibasic calcium phosphate (DCP) at 86%, hydroxypropyl methyl cellulose (HPMC) at 5%, and crospovidone (CPVD) at 9%. The excipient was produced by a wet homogenization/spray drying granulation process. The apparatus used for the production of the excipient is a Co-current atomizer disc type with the disc RPM between 12000-25000 and the inlet temperatures of 180-250° C. After granulation a cyclone separation device was used to remove the fines. Powdered DCP was converted in a mixing chamber into a slurry using deionized water to reach a concentration of 28.7% w/w. In a separate tank crospovidone was mixed with deionized water to give a slurry with a concentration of 15.3% w/w. The crospovidone slurry was added to the DCP slurry and the mixture was stirred, circulated and homogenized for 75 min. To the DCP/CPVD slurry was added a 14.3% w/w HPMC/deionized water slurry and the resulted mixture was stirred, circulated and homogenized for 60 min to form a uniform slurry with a total slurry concentration of 25.0%. The slurry mixture was then spray dried through a rotary nozzle at the motor frequency of 22-28 Hz in the presence of hot air at an outlet temperature of 113-118° C. This constitutes the granule formation step. SEM micrographs of the excipient of Example 1 are seen in FIG. 1. SEM micrographs were recorded using a FEI XL30 ESEM (environmental scanning electron microscope), voltage 5 kV, spot size 2.5, SE detector. The samples were sputtered with Iridium before SEM analysis (sputtering time 40 sec).

The compressibility, aerated bulk density and tapped bulk density of the granular material were measured using a Powder Tester (Hosokawa Micron Corporation) Model PT-S. A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm. D50 value was calculated based on the data collected in a “particle size distribution” measurement. An Air Jet Sieving instrument (Hosokawa Micron System) was used to determine the particle size distribution of the granular material. A set of four sieves (270 mesh, 200 mesh, 100 mesh and 60 mesh) was used. The sieving time for each sieve was 60 sec, while the vacuum pressure was maintained at 10-12 in. H₂O. The sample size was 5 g.

The “loss on drying” (LOD) value was determined using a Mettler Toledo Infrared Dryer LP16. The set temperature was 120° C. and the analysis was stopped when constant weight was reached.

TABLE 1

| Powder Characteristic | Value |
|---|---|
| Angle of repose (°) | 30.1 |
| Aerated Bulk Density (g/cc) | 0.268 |
| Tapped Bulk Density (g/cc) | 0.323 |
| Compressibility (%) | 17.0 |
| Hausner ratio | 1.21 |
| D50 (um) | 131.8 |
| LOD (%) | 1.3 |

### Example 2

High Shear Wet Granulation of Dibasic Calcium Phosphate (89%)-HPMC (2%)-Crospovidone (9%):

179.1 g dibasic calcium phosphate, 4.0 g Hydroxypropyl methylcellulose and 18.1 g crospovidone was placed in a 1 L stainless steel bowl. The bowl was attached to a GMX.01 vector micro high shear mixer/granulator (Vector Corporation). The dry mixture was mixed for 2 minutes at 870 rpm impeller speed and 1000 rpm chopper speed. 35 g of deionized water (“the liquid binder”) was added to the dry blend, drop by drop, using a peristaltic pump at a dose rate of 43 rpm. During the liquid binder addition the impeller speed was 1450 rpm and the chopper speed was 1500 rpm. The wet massing time was 180 seconds maintaining the same impeller and chopper speed as during the liquid addition. Following the granulation, the wet granular material was dried in a tray at 60° C. The resulted granular material (moisture content 2.5%) was screened through a 30 mesh sieve. The yield of the granular material that passed through 30 mesh screen was 123.0 g.

### Example 3

High Shear Wet Granulation of Dibasic Calcium Phosphate (86%)-HPMC (5%)-Crospovidone (9%):

173.0 g dibasic calcium phosphate, 10.1 g Hydroxypropyl methylcellulose and 18.1 g crospovidone was placed in a 1 L stainless steel bowl. The bowl was attached to a GMX.01 vector micro high shear mixer/granulator (Vector Corporation). The dry mixture was mixed for 2 minutes at 870 rpm impeller speed and 1000 rpm chopper speed. 35 g of deionized water (“the liquid binder”) was added to the dry blend, drop by drop, using a peristaltic pump at a dose rate of 43 rpm. During the liquid binder addition the impeller speed was 1450 rpm and the chopper speed was 1500 rpm. The wet massing time was 180 seconds maintaining the same impeller and chopper speed as during the liquid addition. Following the granulation, the wet granular material was dried in a tray at 60° C. The resulted granular material (moisture content 2.0%) was screened through a 30 mesh sieve. The yield of the granular material that passed through 30 mesh screen was 97.3 g. SEM micrographs of this material were recorded using a FEI XL30 ESEM (environmental scanning electron microscope), voltage 5 kV, spot size 2.5, SE detector. The samples were sputtered with Iridium before SEM analysis (sputtering time 40 sec). See FIG. 2.

### Example 4

Powder blend of Dibasic Calcium Phosphate, Hydroxypropyl methylcellulose and Crospovidone:

Predetermined amounts (see Table 2) of Dibasic Calcium Phosphate, Hydroxypropyl methylcellulose and Crospovidone were blended in a 4-L V-blender for two hours.

TABLE 2

| | Dibasic Calcium | Hydroxypropyl | Crospovidone |
|---|---|---|---|
| Example | Phosphate (g) | methylcellulose (g) | (g) |
| 4a | 179.1 | 4.0 | 18.1 |
| 4b | 173.0 | 10.1 | 18.1 |

### Example 5

Granules friability test for the Example 1 excipient and the material obtained by high shear wet granulation as per Examples 3:

75-100 g of granular material was analyzed for particle size distribution and then was loaded in a 4 L V-Blender and tumbled for 2 h. The granular material was collected and analyzed again for particle size distribution. An Air Jet Sieving instrument (Hosokawa Micron System) was used to determine the particle size distribution of the granular material before and after tumbling. A set of four sieves (270 mesh, 200 mesh, 100 mesh and 60 mesh) was used. The sieving time for each sieve was 60 sec, while the vacuum pressure was maintained at 12-14 in. H₂O. The sample size was 5 g.

TABLE 3

| | % Particles with diameter | % Particles with diameter |
|---|---|---|
| | less than 50 microns | less than 50 microns |
| Sample | before tumbling | after tumbling |
| Example 1 | 7.4 | 7.7 |
| Example 3 | 23.4 | 39.9 |

### Example 6

Comparison of Powder Characteristics for Example 1, Example 3 and Example 4b Excipients:

The powder properties of the materials prepared in Example 1, example 3 and Example 4b were measured using a Powder Tester (Hosokawa Micron Corporation) Model PT-S. The Hosokawa Powder tester determines flowability of dry solids in accordance with the proven method of R. L. Carr. A computer which uses the Hosokawa Powder Tester software was used to control the Hosokawa Powder Tester during the measurement operation, enabling simple use and data processing. For measuring the aerated bulk density and tapped bulk density a 50 cc cup was employed. The standard tapping counts for measuring the tapped bulk density were 180 and the tapping stroke was 18 mm.

TABLE 4
Powder Characteristics for DCP(89%)-HPMC(5%)-CPVD(9%) materials
prepared according to Examples 1, 3 and 4b, respectively

| | Example 1 | | Example 3 | | Example 4b* | |
|---|---|---|---|---|---|---|
| Property | Value | Index | Value | Index | Value | Index |
| Angle of repose (deg) | 30.1 | 22.5 | 36.1 | 19.5 | — | — |
| Aerated Bulk Density | 0.268 | | 0.586 | | 0.709 | — |
| (g/cc) | | | | | | |
| Packed Bulk Density | 0.323 | | 0.786 | | 1.181 | — |
| (g/cc) | | | | | | |
| Compressibility (%) | 17 | 18.0 | 25.4 | 15.0 | 40 | — |
| Angle of Spatula | 29.6 | | 34.0 | | — | — |
| Before Impact | | | | | | |
| Angle of Spatula | 23.4 | | 29.6 | | — | — |
| After Impact | | | | | | |
| Angle of spatula (avg) | 26.5 | 24.0 | 31.8 | 22.0 | — | — |
| Uniformity | 3.1 | 23.0 | 3.9 | 23.0 | — | — |
| Total Flowability | | 87.5 | | 79.5 | — | — |
| Index | | | | | | |

*The flowability of the powder bend prepared according to example 4b was extremely poor.

### Example 7

Comparison of Hausner Ratio and Carr's Compressibility Index (%) for Example 1, Example 3 and Example 4b excipients:

Using the aerated and tapped bulk density, Carr's compressibility index and Hausner ratio can be calculated. A value of 20-21% or less for the Carr's compressibility index and a value below 1.25 for the Hausner ratio indicate a material with good flowability.

TABLE 5

| Excipient Brand | Hausner | Compressibility |
|---|---|---|
| Name | Ratio | Index (%) |
| Example 1 | 1.205 | 17.0 |
| Example 3 | 1.341 | 25.4 |
| Example 4b | 1.666 | 40.0 |

### Example 8

Comparison of tablet hardness and tablet disintegration time for placebo tablets prepared using Example 1, the material obtained by high shear wet granulation as per Example 3 and the powder blend obtained as per Example 4b:

Approximately 0.5 g tablets were pressed from the corresponding granular material at various compression forces using a Carver manual press and a 13 mm die. The dwell time was 5 seconds. No lubricant was added. The hardness of the tablets was measured using a Varian, Benchsaver™ Series, VK 200 Tablet Hardness Tester. The values recorded in the table below are an average of three measurements.

TABLE 6

| | Hardness (kp) | | | Disintegration time (sec) | | |
|---|---|---|---|---|---|---|
| Compression | Exam- | Exam- | Exam- | Exam- | Exam- | Exam- |
| Force (lbs-f) | ple 1 | ple 3 | ple 4b | ple 1 | ple 3 | ple 4b |
| 5000 | 9.7 | 4.27 | 3.2 | 150 | 210 | 240 |
| 10000 | 16.2 | 6.35 | 5.57 | 167 | 225 | 205 |
| 15000 | 20.1 | — | — | 150 | — | — |

### Example 9

Preparation of 33.3% Diclofenac Sodium Immediate Release Tablets Using the Excipient Prepared as per Example 1:

100 g of Diclofenac Sodium was blended with 197 g example 1 excipient in a V-blender at 20 rpm for 15 min. 3 g of Magnesium Stearate was added to the resulting blend and the mixture was blended for an additional 2 minutes at 20 rpm. Approximately 0.5 g tablets were pressed from the final blend at various compression forces using a Carver manual press and a 13 mm die. The dwell time was 5 seconds. The hardness of the tablets was measured using a Varian, Benchsaver™ Series, VK 200 Tablet Hardness Tester. The disintegration experiments were performed with a Distek Disintegration System 3100, using 900 mL deionized water at 37±0.5° C. degrees Celsius.

Dissolution studies were performed with an USP Apparatus 2 Distek Dissolution System 2100A using 900 mL sodium phosphate buffer pH 6.8 as dissolution medium, at 37±0.5° C. The paddle rotation was 50 rpm. Samples were taken at 5, 10, 15, 20, 30, 45 and 60 minutes, respectively. The amount of Diclofenac Sodium dissolved was determined from UV absorbance at about 276 nm on filtered portions of the solutions under test, suitable diluted with medium, in comparison with a standard solution containing Diclofenac Sodium.

TABLE 7
Tablet Hardness as a function of the compression force
for tablets prepared according to Example 9

| Compression Force | Hardness |
|---|---|
| (lbs-force) | (kp) |
| 3000 | 8.6 |
| 5000 | 8.5 |
| 7000 | 8.25 |
| 10000 | 8.9 |

The disintegration time of the tablets prepared according to Example 9 was between 20 minutes and 30 minutes. The dissolution profile for Diclofenac Sodium from tablets prepared at 5000 lbs-force according to Example 9 is shown in FIG. 6.

Having described the invention in detail, those skilled in the art will appreciate that modifications may be made of the invention without departing from its' spirit and scope. Therefore, it is not intended that the scope of the invention be limited to the specific embodiments described. Rather, it is intended that the appended claims and their equivalents determine the scope of the invention.

Unless otherwise noted, all percentages are weight/weight percentages.

1. A composition comprising:
about 75% to about 98% dibasic calcium phosphate;
about 1% to about 10% at least one binder; and
about 1% to about 20% at least one disintegrant,
wherein the dibasic calcium phosphate, binder and disintegrant form substantially homogeneous spherical particles in which the dibasic calcium phosphate, binder and disintegrant are indistinguishable when viewed with a SEM.

2. The composition of claim 1 wherein the composition includes:
about 80% to about 90% dibasic calcium phosphate;
about 2% to about 8% at least one binder; and
about 3% to about 12% at least one disintegrant.

3. The composition of claim 1 wherein the composition includes:
about 85% to about 93% dibasic calcium phosphate;
about 2% to about 5% at least one binder; and
about 10% at least one disintegrant.

4. The composition of claim 1 wherein the binder includes hydroxypropyl methylcellulose and the disintegrant includes cross-linked polyvinylpyrrolidone.

5. The composition of claim 1 wherein the excipient is formed by spraying an aqueous slurry comprised of the dibasic calcium phosphate, binder and disintegrant.

6. A method of making an excipient comprising:
forming a dibasic calcium phosphate slurry;
forming a binder slurry;
forming a disintegrant slurry;
homogenizing the dibasic calcium phosphate slurry and the disintegrant slurry to form a DCP/disintegrant slurry;
adding the binder slurry to the DCP/disintegrant slurry;
and spray dry granulating the final slurry to form particles of excipient,
wherein the dibasic calcium phosphate, binder and disintegrant are indistinguishable when viewed with a SEM, thereby forming substantially homogeneous spherical particles.

7. The method of claim 6 wherein:
about 75% to about 98% dibasic calcium phosphate;
about 1% to about 10% at least one binder; and
about 1% to about 20% at least one disintegrant.

8. The method of claim 6 comprising:
about 80% to about 90% dibasic calcium phosphate;
about 2% to about 8% at least one binder; and
about 3% to about 12% at least one disintegrant.

9. The method of claim 6 comprising:
about 85% to about 93% dibasic calcium phosphate;
about 2% to about 5% at least one binder; and
about 10% at least one disintegrant.

10. The method of claim 6 wherein the binder includes hydroxypropyl methylcellulose and the disintegrant includes cross-linked polyvinylpyrrolidone.

11. A method of making an excipient comprising:
forming a dibasic calcium phosphate slurry;
forming a hydroxypropyl methylcellulose slurry;
forming a cross-linked polyvinylpyrrolidone slurry;
homogenizing the dibasic calcium phosphate slurry and the cross-linked polyvinylpyrrolidone slurry to form a DCP/cross-linked polyvinylpyrollidone slurry;
adding the hydroxypropyl methylcellulose slurry to the DCP/cross-linked polyvinylpyrollidone slurry; and
spray dry granulating the final slurry to form particles of excipient,
wherein the dibasic calcium phosphate, hydroxypropyl methylcellulose and cross-linked polyvinylpyrrolidone are indistinguishable when viewed with a SEM, thereby forming substantially homogeneous spherical particles.

12. The method of claim 11 comprising:
about 75% to about 98% dibasic calcium phosphate;
about 1% to about 10% hydroxypropyl methylcellulose; and
about 1% to about 20% at least one cross-linked polyvinylpyrrolidone.

13. The method of claim 11 comprising:
about 80% to about 90% dibasic calcium phosphate;
about 2% to about 8% hydroxypropyl methylcellulose; and
about 3% to about 12% at least one cross-linked polyvinylpyrrolidone.

14. The method of claim 11 comprising:
about 85% to about 93% dibasic calcium phosphate;
about 2% to about 5% hydroxypropyl methylcellulose; and
about 10% at least one cross-linked polyvinylpyrrolidone.

15. A pharmaceutical tablet comprising:
at least one active pharmaceutical ingredient; and
an excipient of substantially homogeneous particles including:
a) dibasic calcium phosphate;
b) at least one binder; and
c) at least one disintegrant.

16. The tablet of claim 15 wherein the excipient includes:
about 75% to about 98% dibasic calcium phosphate;
about 1% to about 10% at least one binder; and
about 1% to about 20% at least one disintegrant.

17. The tablet of claim 15 wherein the excipient includes:
about 80% to about 90% dibasic calcium phosphate;
about 2% to about 8% at least one binder; and
about 3% to about 12% at least one disintegrant.

18. The tablet of claim 15 wherein the excipient includes:
about 85% to about 93% dibasic calcium phosphate;
about 2% to about 5% at least one binder; and
about 10% at least one disintegrant.

19. The tablet of claim 15 wherein the binder includes hydroxypropyl methylcellulose and the disintegrant includes cross-linked polyvinylpyrrolidone.

20. A method of making a pharmaceutical tablet comprising:
mixing at least one active pharmaceutical ingredient with an excipient of substantially homogeneous particles including:
a) dibasic calcium phosphate;
b) at least one binder; and
c) at least one disintegrant to form a mixture; and
compressing the mixture to form a tablet.

21. The method of claim 20 wherein the excipient includes:
about 75 to about 98% dibasic calcium phosphate;
about 1% to about 10% at least one binder; and
about 1% to about 20% at least one disintegrant.

22. The method of claim 20 wherein the excipient includes:
about 80% to about 90% dibasic calcium phosphate;
about 2% to about 8% at least one binder; and
about 3% to about 12% at least one disintegrant.

23. The method of claim 20 wherein the excipient includes:
about 85% to about 93% dibasic calcium phosphate;
about 2% to about 5% at least one binder; and
about 10% at least one disintegrant.

24. The method of claim 20 wherein the binder includes hydroxypropyl methylcellulose and the disintegrant includes cross-linked polyvinylpyrrolidone.
